(19) United States
(12) Patent Application Publication
(10) Pub. No.: US 2007/0270563 A1
(43) Pub. Date: Nov. 22, 2007

(54) CyclicOrganosilicon Compounds and the Use Thereof

(75) Inventors: Wolfgang Ziche, Burghausen (DE); Ernst Selbertinger, Burghausen (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 11/573,734

(22) PCT Filed: Aug. 4, 2005
(86) PCT No.: PCT/EP05/08460

§ 371 Date: Feb. 15, 2007

(30) Foreign Application Priority Data
Aug. 19, 2004 (DE) 10 2004 040 314.7

(51) Int. Cl. C08G 77/04; C07D 517/00

(52) U.S. Cl. 528/29; 548/100

(57) ABSTRACT

Siloxane-urea copolymers are easily prepared by reacting a bis- or poly[azasilacyclopentyl]-terminated compound with a hydroxyl-functional compound followed by reaction with di- or polyisocyanate. When a bis[hydroxyl]-functional compound is used, the resulting polymer may be reacted with additional di- or polyisocyanate, optionally in the presence of additional isocyanate-reactive compounds such as chain extenders.

The invention relates to cyclic organosilicon compounds and to the use thereof, in particular in a process for the preparation of copolymers.

The preparation of siloxane-urea block copolymers is known. Reference may be made, for this, for example, to EP-A 250 248. Aminoalkyl-functional siloxanes are prepared as starting materials via equilibrium reactions. However, as described in DE-A1 101 37 855, there are disadvantages to the processes disclosed in EP-A 250 248 for the preparation of such siloxanes: the reaction is lengthy, special catalysts are necessary, these catalysts have to be deactivated in the product, which leads to yellowing when the products are used, and the product comprises siloxane rings. DE-A1 101 37 855 discloses a better synthesis given the use of special cyclic silazanes. A disadvantage of all known processes is that the aminoalkyl-functional siloxanes have to be reacted with, with reference to the weight, very little diisocyanate afterwards to give the desired block copolymers. In this connection, high local concentrations of highly reactive isocyanate groups cannot be avoided on mixing the reaction components. This results in side reactions, such as, e.g., the formation of biurets, which has a negative effect on the properties of the polymeric product.

A subject matter of the invention are cyclic organosilicon compounds of the formula (I) 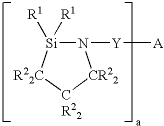
in which
A represents a di- or polyvalent organic radical,
a, corresponding to the valency of the radical A, represents a value ≧2,
R¹ can be identical or different and represents a monovalent organic radical,
R² can be identical or different and represents a hydrogen atom or an optionally substituted monovalent hydrocarbon radical,
Y represents an —SiR₂—Z—NR³—C(═O)—NH— radical or a —C(═O)—NH— radical,
Z represents a divalent hydrocarbon radical,
R can be identical or different and represents a monovalent organic radical and
R³ represents a hydrogen atom or an optionally substituted monovalent hydrocarbon radical.

The radical A preferably concerns di- or polyvalent hydrocarbon radicals optionally substituted by fluorine or chlorine in which methylene units not adjacent to one another can be replaced by —O—, —COO—, —OCO—, —CO—NH or —OCOO— groups, particularly preferably divalent alkylene radicals with 1 to 60 carbon atoms, in particular divalent alkylene radicals with 6-24 carbon atoms.

Examples of radicals A are alkylene radicals, such as the methylene, ethylene, n-propylene, isopropylene, n-butylene, isobutylene, tert-butylene, n-pentylene, isopentylene, neopentylene or tert-pentylene radical, hexylene radicals, such as the n-hexylene radical, heptylene radicals, such as the n-heptylene radical, octylene radicals, such as the n-octylene radical and isooctylene radicals, such as the 2,2,4-trimethyl-pentylene radical, nonylene radicals, such as the n-nonylene radical, decylene radicals, such as the n-decylene radical, or dodecylene radicals, such as the n-dodecylene radical; alkenylene radicals, such as the vinylene radical and the allylene radical; cycloalkylene radicals, such as isophoronylene, 4,4′-dicyclohexylmethylene, cyclopentylene, cyclo-hexylene or cycloheptylene radicals and methylcyclo-hexylene radicals; arylene radicals, such as the phenylene radical and the naphthylene radical; alkarylene radicals, such as o-, m- or p-tolylene radicals, the 4,4′-diphenylmethylene radical, xylylene radicals and ethylphenylene radicals; aralkylene radicals, such as the benzylene radical, the α-phenylethylene radical and the β-phenylethylene radical; and divalent polymer radicals, such as polyether radicals and polyurethane radicals.

The preferred value for a, the number of the aza rings in the compound of the formula (I), is 2.

The radical R¹ preferably concerns, independently of one another, optionally substituted hydrocarbon radicals which can be interrupted by heteroatoms and/or can be bonded via heteroatoms to the silicon atom.

Examples of radicals R¹ are SiC-bonded hydrocarbon radicals, such as, e.g., alkyl radicals, such as the methyl, ethyl, n-propyl, isopropyl, 1-n-butyl, 2-n-butyl, isobutyl, tert-butyl, n-pentyl, isopentyl, neopentyl or tert-pentyl radical; hexyl radicals, such as the n-hexyl radical; heptyl radicals, such as the n-heptyl radical; octyl radicals, such as the n-octyl radical and isooctyl radicals, such as the 2,2,4-trimethylpentyl radical; nonyl radicals, such as the n-nonyl radical; decyl radicals, such as the n-decyl radical; dodecyl radicals, such as the n-dodecyl radical; octadecyl radicals, such as the n-octadecyl radical; cycloalkyl radicals, such as the cyclopentyl, cyclohexyl or cycloheptyl radical and methylcyclohexyl radicals; alkenyl radicals, such as the vinyl, 1-propenyl and 2-propenyl radical; aryl radicals, such as the phenyl, naphthyl, anthryl and phenanthryl radical; alkaryl radicals, such as o-, m- or p-tolyl radicals, xylyl radicals and ethylphenyl radicals; and aralkyl radicals, such as the benzyl radical, the α-phenylethyl radical and the β-phenylethyl radical, SiC-bonded substituted hydrocarbon radicals, such as, e.g., haloalkyl radicals, such as the 3,3,3-trifluoro-n-propyl radical, the 2,2,2,2′,2′,2′-hexafluoroiso-propyl radical or the heptafluoroisopropyl radical, haloaryl radicals, such as the o-, m- and p-chloro-phenyl radical, or aminoalkyl radicals, such as the aminomethyl radical and the 3-aminopropyl radical, optionally substituted hydrocarbon radicals which are bonded via heteroatoms to the silicon atom, such as, e.g., alkoxy radicals, such as the methoxy, ethoxy and methoxyethoxy radical, acyloxy radicals, such as the acetoxy radical, oxime radicals, such as the methyl ethyl ketoxime radical, and alkylamino radicals, such as the cyclohexylamino radical.

The radical R¹ concerns particularly preferably SiC-bonded hydrocarbon radicals and SiOC-bonded alkoxy radicals, very particularly preferably the methyl, ethyl, phenyl, ethoxy and methoxy radical, in particular the methyl and methoxy radical.

The radical R² preferably concerns a hydrogen atom and a methyl or ethyl radical, particularly preferably a hydrogen atom or a methyl radical, in particular a hydrogen atom.

Examples of radicals R² are the examples given for R¹ of SiC-bonded optionally substituted hydrocarbon radicals.

The radical Z preferably concerns alkylene radicals, the methylene and propylene radical being particularly preferred.

Examples of the radical Z are the divalent hydrocarbon radicals given for the radical A.

The radical R preferably concerns alkyl, aryl or alkoxy radicals, alkyl radicals being particularly preferred, in particular the methyl radical.

Examples of the radical R are the examples given for the radical R¹.

The radical R³ preferably concerns alkyl radicals and a hydrogen atom, a hydrogen atom being particularly preferred.

Examples of the radical R³ are the examples given for the radical R².

Y preferably concerns the —SiR₂—Z—NR³—C(═O)—NH— radical with R, R³ and Z the same as one of the meanings given above, particularly preferably the —SiR₂—Z—NH—C(═O)—NH— radical, in particular —SiR₂—(CH₂)₃—NH—C(═O)—NH—.

Examples of the organosilicon compounds of the formula (I) according to the invention are
[cyclo-(Me₂Si—(CH₂)₃—N)—SiMe₂—(CH₂)₃—NH—C(═O)—NH]₂(C₆H₃)—(CH₃ ),
[cyclo-(Me₂Si—(CH₂)₃—N)—SiMe₂—(CH₂)₃—NH—C(═O)—NH]₂(CH₂)₆,
[cyclo-(Me₂Si—(CH₂)₃—N)—SiMe₂—(CH₂)₃—NH—C(═O)—NH]₂(C₆H₇)—(CH₃)₂,
[cyclo-(Me₂Si—(CH₂)₃—N)—SiMe₂—(CH₂)₃—NH—C (═O)—NH]₂(C₆H₄)₂—CH₂,
[cyclo (Me₂Si—(CH₂)₃—N)—SiMe₂—(CH₂)₃—NH—C(═O)—NH]₂(C₆H₁₀)₂—CH₂,
[cyclo-((MeO)₂Si—(CH₂)₃—N)—C(═O)—NH]₂(C₆H₃)(CH₃),
[cyclo-((MeO)₂Si—(CH₂)₃—N)—C(═O)—NH]₂(CH₂)₆,
[cyclo-((MeO)₂Si—(CH₂)₃—N)—C(═O)—NH]₂(C₆H₇)(CH₃)₂,
[cyclo-((MeO)₂Si—(CH₂)₃—N)—C(═O)—NH]₂(C₆H₄)₂CH₂ and
[cycl-((MeO)₂Si—(CH₂)₃—N)—C(═O)—NH]₂(C₆H₁₀)₂CH₂.

The organosilicon compounds according to the invention are moisture-sensitive compounds and may be liquid or solid, preferably liquid, at ambient temperature and the pressure of the ambient atmosphere, thus between 900 and 1100 hPa.

If the organosilicon compounds according to the invention are liquids, they have a viscosity preferably of 20 to 100 000 mm²/s at 25° C.

The organosilicon compounds of the formula (I) according to the invention can now be prepared according to any process known in silicon chemistry. The organosilicon compounds according to the invention are preferably prepared by reaction of azasilacyclopentane with polyisocyanate.

A further subject matter of the invention is a process for the preparation of cyclic organosilicon compounds of the formula (I), characterized in that azasilacyclopentane is reacted with polyisocyanate.

In the context of the present invention, the term “poly” is to embrace polymeric, oligomeric and dimeric compounds.

In the process according to the invention, the azasilacyclopentane and polyisocyanate are preferably reacted with the exclusion of water and moisture.

The process according to the invention is carried out at a temperature preferably of 0 to 100° C., particularly preferably of 20 to 50° C., and a pressure of the ambient atmosphere, thus 900 to 1100 hPa.

In the process according to the invention for the preparation of compounds of the formula (I), azasilacyclopentane is used in the stoichiometric ratio to the isocyanate groups of the polyisocyanate used of preferably 0.9:1 to 1:0.9, particularly preferably 1:1.

If desired, the process according to the invention can be carried out in the presence of polar organic solvents, such as acetone, tetrahydrofuran or isopropanol. Preferably, however, no polar organic solvent is used. If polar solvents are used in the process according to the invention, these do not necessarily have to be removed before the further processing of the compounds (I) according to the invention.

The process according to the invention for the preparation of cyclic organosilicon compounds of the formula (I) can be carried out both batchwise or continuously.

The azasilacyclopentane used according to the invention is a commercial product or can be prepared according to processes standard in silicon chemistry, such as, e.g., disclosed in the abovementioned DE-A1 10137855.

The organosilicon compounds according to the invention can now be used for all purposes for which cyclic organosilicon compounds were able to be used hitherto.

In particular, they are suitable for the preparation of copolymers.

A further subject matter of the invention is a process for the preparation of copolymers, characterized in that, in a first step,
cyclic organosilicon compounds of the formula (I) are reacted with compounds (2) exhibiting hydroxyl groups and, optionally, in a second step,
   the reaction product thus obtained is reacted with polyisocyanate (3).

In the process according to the invention for the preparation of copolymers, any organic and organosilicon hydroxyl compound can be used as compound (2) exhibiting hydroxyl groups.

Preferably, the compounds (2) comprise two hydroxyl groups.

The compounds (2) exhibiting hydroxyl groups used according to the invention are preferably alcohols and organosilicon compounds, particularly preferably organosilicon compounds.

If organosilicon compounds are used as compound (2) used according to the invention, those are preferably concerned which comprise units of the formula
R⁴_{b}(OH)_{c}SiO_{4-b-c/2} (II)
in which
R⁴ can be identical or different and has one of the meanings given for R²,
b is 1, 2 or 3 and
c is 0, 1 or 2,
   with the proviso that the sum b+c is less than or equal to 4 and, per molecule, at least one Si-bonded hydroxyl group is present.

The organosilicon compounds used according to the invention can be both silanes, i.e. compounds of the formula (II) with b+c=4, and siloxanes, i.e. compounds from units of the formula (II) with b+c<3. Preferably, the organosilicon compounds used according to the invention are organopolysiloxanes, in particular essentially linear organopolysiloxanes, consisting of units of the formula (II).

Examples of the radical R⁴ are the examples given for R¹ of SiC-bonded optionally substituted hydrocarbon radicals.

The radical R⁴ preferably concerns hydrocarbon radicals, particularly preferably hydrocarbon radicals with 1 to 4 carbon atoms, in particular the methyl radical.

Preferably, b has a value of 2.

Preferably, c has a value of 0 or 1.

Examples of compound (2) used according to the invention are mono- or polyhydric alcohols, such as, e.g., methanol, ethanol, n-propanol, isopropanol, 1,2-propanediol, 1,3-propanediol, 1-butanol, 2-butanol, tert-butanol, 1,4-butanediol, 1-pentanol, 2-pentanol, 3-pentanol, 1,5-pentanediol, 1-hexanol, cyclohexanol, 1-heptanol, 1-octanol, 1-decanol, lauryl alcohol, myristyl alcohol, stearyl alcohol, benzyl alcohol, diethylene glycol, triethylene glycol, dipropylene glycol, ethylene glycol monomethyl ether, ethylene glycol monoethyl ether, ethylene glycol monobutyl ether, diethylene glycol monomethyl ether, diethylene glycol monobutyl ether, α,ω-hydroxy-terminated polyethylene glycols and α,ω-hydroxy-terminated polypropylene glycols, and also organopolysiloxanes exhibiting hydroxyl groups, such as α,ω-silanol-terminated polydiorganosiloxanes, preferably α,ω-silanol-terminated polydimethylsiloxanes, and silanols, such as diphenylsilanediol.

If organosilicon compounds are concerned as compound (2) used according to the invention, these have a viscosity preferably of 5 to 500 000 nm²/s, particularly preferably 50 to 5000 mm²/s, in each case at 25° C.

In the first step of the process according to the invention, the stoichiometric ratio of the hydroxyl groups in compound (2) to the aza rings in the cyclic organosilicon compound of the formula (I) is preferably 0.9:1 to 1:0.9, particularly preferably 1:1.

In the first step of the process according to the invention for the preparation of copolymers, the compounds of the formula (I) according to the invention and compound (2) are preferably reacted with the exclusion of water and moisture.

The first step of the process according to the invention for the preparation of copolymers is carried out at temperatures preferably of 0 to 200° C., particularly preferably at 20 to 150° C., and the pressure of the ambient atmosphere, thus approximately 900 to 1100 hPa.

If desired, the first step of the process according to the invention for the preparation of copolymers can be carried out in the presence of polar organic solvents, such as acetone, tetrahydrofuran or isopropanol. Preferably, no polar organic solvent is used; in this connection, the reaction mass is preferably maintained at a temperature at which the viscosity thereof is not greater than 10 000 Pa.s. If polar solvents are used, these do not necessarily have to be removed before the second step, optionally carried out, of the process according to the invention.

Examples of the polyisocyanate (3) used in the second step of the process according to the invention optionally carried out are hexylene diisocyanate, 4,4′-methylenedicyclohexylene diisocyanate, 4,4′-methylenediphenylene diisocyanate, 1,3-di-azetidine-2,4-dione bis[(4,4′-methylenedicyclohexyl) diisocyanate], 1,3-diazetidine-2,4-dione bis-[(4,4′-methylenediphenyl) diisocyanate], tris(iso-cyanatohexyl) isocyanurate, tetramethylxylylene diiso-cyanate and isophorone diisocyanate.

The polyisocyanates (3) used according to the invention are preferably diisocyanates, such as hexylene diiso-cyanate, 4,4′-methylenedicyclohexylene diisocyanate, 4,4′-methylenediphenylene diisocyanate, 1,3-di-azetidine-2,4-dione bis[(4,4′-methylenedicyclohexyl) diisocyanate], 1,3-diazetidine-2,4-dione bis-[(4,4′-methylenediphenyl) diisocyanate], tetramethyl-xylylene diisocyanate and isophorone diisocyanate, particularly preferably hexylene diisocyanate, 4,4′-methylenedicyclohexylene diisocyanate, 4,4′-methylenediphenylene diisocyanate, tetramethyl-xylylene diisocyanate and isophorone diisocyanate, in particular hexylene diisocyanate, 4,4′-methylenedi-cyclohexylene diisocyanate and isophorone diisocyanate.

If the second step of the process according to the invention is carried out, compound (3) is used in the stoichiometric ratio of its isocyanate groups to the amino groups of the reaction product obtained in the first step of the process according to the invention preferably of 0.9:1 to 1:0.9, particularly preferably of 1:1.

If desired, use may be made, in the second step, optionally carried out, of the process according to the invention for the preparation of copolymers, of “chain extenders” known in polyurethane chemistry to a person skilled in the art. However, this is not preferred.

Examples of chain extenders which can be used in the second stage of the process according to the invention are difunctional organic compounds, such as, e.g., diols and diamines.

In the second step, optionally carried out, of the process according to the invention for the preparation of copolymers, the reaction product obtained in the first stage is reacted with the polyisocyanate (3), preferably with the exclusion of water and moisture.

The second step of the process according to the invention for the preparation of copolymers is carried out at temperatures preferably of 0 to 200° C., particularly preferably at 20 to 150° C., and the pressure of the ambient atmosphere, thus approximately 900 to 1100 hPa.

If desired, the second step of the process according to the invention for the preparation of copolymers can be carried out in the presence of polar organic solvents, such as acetone, tetrahydrofuran or isopropanol. Preferably, no polar organic solvent is used; in this connection, the reaction mass is preferably maintained at a temperature at which the viscosity thereof is not greater than 10 000 Pa.s.

The second step of the process according to the invention is then carried out in particular if organosilicon compounds of the formula (I) with Y=—SiR₂—Z—NR³—C(═O)—NH— are used as organosilicon compounds of the formula (I).

The process according to the invention for the preparation of copolymers can be carried out both batchwise or continuously. If desired, the preparation according to the invention of the cyclic organosilicon compound of the formula (I) can be carried out directly as initial stage in the one-pot process, e.g. in an extrusion process.

The copolymers prepared according to the invention can now be isolated according to processes known per se, such as, e.g., removal by means of molecular distillation of the solvent optionally used.

The copolymers prepared according to the invention are preferably thermoplastic elastomers and have a number-average molecular weight Mₙ of >100 000, preferably >500 000.

The copolymers prepared according to the invention can be used for all purposes for which urea copolymers have also been used hitherto. In particular, they are suitable as additive in plastics processing (e.g., extrusion, injection molding, fiber spinning), as functional additive in other plastics, as thermoplastic materials which can be processed by extrusion, coextrusion and injection molding to give profiles, films and components, in solution or dispersion for the coating of surfaces of all kinds, such as plastics, metals, wood or textiles.

The cyclic organosilicon compounds of the formula (I) according to the invention have the advantage that they react with hydroxyl groups in a fast reaction without the production of byproducts. In addition, they are simple to prepare.

The process according to the invention for the preparation of organosilicon compounds of the formula (I) has the advantage that it is simple, is generally free from solvent, is free from catalyst and above all is fast, which makes possible a continuous method.

The process according to the invention for the preparation of copolymers has the advantage that no derivatization of base polymers expensive in terms of time and processing is necessary.

In the following examples, all statements of parts and percentages, unless otherwise specified, refer to the weight. Unless otherwise specified, the following examples are carried out at a pressure of the ambient atmosphere, thus at approximately 1000 hPa, and at ambient temperature, thus approximately 20° C., or a temperature which ensues on mixing together the reactants at ambient temperature without additional heating or cooling. All viscosity statements cited in the examples are with reference to a temperature of 25° C.

The Shore A hardness is determined according to DIN (Deutsche Industrie Norm) 53505 (Edition August 2000).

Tensile strength, elongation at break and modulus (tension at 100% elongation) were determined according to DIN 53504 (Edition May 1994) on test specimens of S2 form.

In the following, the abbreviation Me is used for the methyl radical.

### EXAMPLE 1

The reaction described subsequently was carried out in a corotating W&P twin-screw extruder (25 mm screw diameter, L/D =40):
2 molar equivalents of 2,2-dimethoxy-l-aza-2-silacyclo-pentane are mixed at 50° C. in the first zone (length L/D=4) of the extruder with 1 molar equivalent of 1,6-diisocyanatohexane. In the second zone of the extruder, one molar equivalent of an α,ω-hydroxy-terminated polydimethylsiloxane with a molecular weight M_{w} of 3000 is metered in. A moisture-crosslinkable transparent thermoplastic siloxane-urea copolymer is obtained which was extruded to give films with a thickness of 2 mm. The films thus obtained were
a) stored for a period of 7 days at 25° C. with the exclusion of moisture (dry storage) or
b) stored for a period of 7 days at 25° C. in water (water storage)

and the mechanical parameters were determined. The results are found in table 1.

TABLE 1

| | | Tensile | Elongation | Tensile modulus |
|---|---|---|---|---|
| | Hardness | strength | at break | at 100% |
| | [Shore A] | [MPa] | [%] | [MPa] |
| Dry | 47 | 0.96 | 383 | 0.87 |
| storage | | | | |
| Storage | 58 | 1.99 | 441 | 1.66 |
| in water | | | | |

### EXAMPLE 2

2 molar equivalents of the silaza ring H₂N—(CH₂)₃—SiMe₂— cyclo-(N—(CH₂)₃—SiMe₂) are reacted with 4,4′-methylene-bis(cyclohexyl isocyanate) without solvent at 60° C. and with good stirring until the characteristic band of the N═C═O group could no longer be detected in the IR spectrum. An organosilicon compound of the formula
[cyclo-(Me₂Si—(CH₂)₃—N)—SiMe₂—(CH₂)₃—NH—C(═O)—NH]₂(C₆H₁₀)₂—CH₂
is obtained.

One molar equivalent of the urea thus obtained is kneaded at 23° C. in a heatable IKA laboratory kneader with two molar equivalents of an α,ω-hydroxy-terminated polydimethylsiloxane with a molecular weight M_{w} of 3000. The temperature is increased to 150° C. and one molar equivalent of 4,4′-methylenebis(cyclohexyl isocyanate) is added. Kneading is carried out for a further 15 minutes for homogenization. A colorless transparent thermoplastic siloxane-urea copolymer is obtained which was extruded to give films with a thickness of 2 mm.

The mechanical parameters were determined. The results are found in table 2.

TABLE 2

| | | Tensile | Elongation | Tensile modulus |
|---|---|---|---|---|
| | Hardness | strength | at break | at 100% |
| | [Shore A] | [MPa] | [%] | [MPa] |
| Example 2 | 45 | 4.1 | 350 | 0.9 |

### EXAMPLE 3

Two molar equivalents of the silaza ring H₂N—(CH₂)₃—SiMe₂-cyclo-(N—(CH₂)₃—SiMe₂) are reacted with 4,4′-methylenebis(cyclohexyl isocyanate) as described in example 2 without solvent to give a viscous colorless urea. One molar equivalent of the urea is kneaded at 23° C. in a heatable IKA laboratory kneader with two molar equivalents of an α,ω-hydroxy-terminated polydimethylsiloxane with a molecular weight M_{w} of 3000. 0.25 molar equivalent of 1,2-diaminoethane is incorporated as additional chain-extending diamine. The temperature is increased to 110° C. and 1.25 molar equivalents of 4,4′-methylenebis(cyclohexyl isocyanate) are added portionwise; the temperature is increased to 160° C. in the process. Kneading is carried out for a further 15 minutes for homogenization. A colorless transparent thermoplastic siloxane-urea copolymer is obtained which was extruded to give films with a thickness of 2 mm. The mechanical parameters were determined. The results are found in table 3.

TABLE 3

| | | Tensile | Elongation | Tensile modulus |
|---|---|---|---|---|
| | Hardness | strength | at break | at 100% |
| | [Shore A] | [MPa] | [%] | [MPa] |
| Example 3 | 51 | 4.5 | 320 | 1.1 |

1.-8. (canceled)

9. A cyclic organosilicon compound of the formula (I) 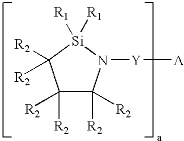
in which
A is a di- or polyvalent organic radical,
a, corresponding to the valency of the radical A, is >2,
R¹ are identical or different monovalent organic radicals,
R² are identical or different and are hydrogen or optionally substituted monovalent hydrocarbon radicals,
Y is an —SiR₂—Z—NR³—C(═O)—NH— radical or a —C(═O)—NH— radical,
Z is a divalent hydrocarbon radical,
R are identical or different monovalent organic radicals, and
R³ is hydrogen or an optionally substituted monovalent hydrocarbon radical.

10. The organosilicon compound of claim 9, wherein a is equal to 2.

11. The organosilicon compound of claim 9, wherein Y is the —SiR₂—Z—NR³—C(═O)—NH radical.

12. A process for the preparation of the cyclic organosilicon compound of claim 1, wherein an azasilacyclopentane is reacted with a polyisocyanate.

13. A process for the preparation of a copolymer, comprising, in a first step, reacting cyclic organosilicon compounds of the formula (I) with compound(s) (2) bearing hydroxyl groups, and, optionally, in a second step, reacting the reaction product thus obtained with polyisocyanate (3).

14. The process of claim 13, wherein compound(s) (2) are alcohols or hydroxyl-functional organosilicon compounds.

15. The process of claim 13, wherein compound(s) (2) are organosilicon compounds comprising units of the formula
R⁴_{b}(OH)_{c}SiO_{4-b-c/2} (II),
in which
R⁴ are identical or different and are hydrogen or optionally substituted monovalent hydrocarbon radicals,
b is 1, 2 or 3, and
c is 0, 1 or 2,
with the proviso that the sum b +c is less than or equal to 4 and, per molecule, at least one Si-bonded hydroxyl group is present.

16. The process of claim 13, wherein compound(s) (2) bear two hydroxyl groups.
